(19) United States
(12) Patent Application Publication
(10) Pub. No.: US 2010/0266649 A1
(43) Pub. Date: Oct. 21, 2010

(54) Gel Technology Suitable for Use in Cosmetic Compositions

(75) Inventors: Prithwiraj Maitra, Randolph, NJ (US); Steven E. Brown, New Windsor, NY (US); John R. Glynn, JR., Ridgewood, NJ (US); Jason Rothouse, Mahwah, NJ (US); John C. Brahms, Morris Plains, NJ (US); Michael J. Fair, Ridgewood, NJ (US)

Correspondence Address: AVON PRODUCTS, INC., AVON PLACE, SUFFERN, NY, 10901, US

(73) Assignee: AVON PRODUCTS, INC., Suffern, NY (US)

(21) Appl. No.: 12/747,469

(22) PCT Filed: Nov. 19, 2008
(86) PCT No.: PCT/US08/84017

§ 371 Date: Jun. 10, 2010

Related U.S. Application Data
(60) Provisional application No. 61/016,967, filed on Dec. 27, 2007.

(51) Int. Cl. A61K 8/02; A61K 47/04; A61Q 1/04; A61Q 1/10; A61Q 1/02; A61Q 5/00; A61Q 17/04

(52) U.S. Cl. 424/401; 424/59; 424/78.03; 424/617; 424/691; 424/724

(57) ABSTRACT

A gel system comprising a fractal network of nanoparticles and macroscopic particles is disclosed. The gel system is capable of forming an “optical gel” effective to blurrfine lines and wrinkles as a consequence of the size domain differences between the fractal particles and the macroscopic particles. Cosmetic compositions comprising such gels and methods for their use are also disclosed.

### RELATED APPLICATIONS

This application claims priority to International Application Serial No. PCT/US08/83490 filed Nov. 14, 2008, which claims priority U.S. Provisional Patent Application Ser. No. 61/014,846, filed Dec. 17, 2007, the contents of which are hereby incorporated by reference in their entirety.

### FIELD OF THE INVENTION

The present invention relates to gel technology comprising macroscopic particles dispersed/suspended/interspersed within a network of fractal particles, more particularly to cosmetic compositions containing such gels to obtain efficient optical blurring of wrinkles, fine lines, pores, skin imperfections, and the like, and most particularly to such cosmetic compositions in which the fractal network is a fractal gel.

### BACKGROUND OF THE INVENTION

A number of methods have been developed to reduce wrinkles and minimize fine lines. Some of these methods include active ingredients such as antioxidants; agents that act by neurotransmission inhibition in nerve cells such as botulinum toxin (Botox™) (Allergan, Irvine, Calif.), thereby relaxing contracted muscles; agents that accelerate the cell renewal process such as hydroxy and fruit acids like retinoic acid; emollients such as shea butter; skin plumpers such as hyaluronic acid; fillers such as collagen; light-diffusing pigments and microspheres which create the illusion that wrinkles have disappeared. Other methods have been developed to reduce the appearance of pores, skin surface unevenness and imperfections and the like. Some of these methods include skin lightening agents, filling and camouflaging the skin.

Unfortunately, many cosmetic foundations and make-ups actually accentuate wrinkles and fine lines due to migration of the pigments into the wrinkle crevices. Other products cover skin imperfections but create an unnatural, caked-on appearance. Others, such as mica, reflect rather than diffuse and scatter light, thereby resulting in an unnatural shiny appearance. Additionally, some of these methods are not immediate, requiring days and weeks of continued use to see effects. Others are invasive, requiring injections, patent discomfort, and may entail redness, swelling and other side effects.

Novel, safe and effective topical “optical blurring” technology to treat wrinkles and skin imperfections are needed. Therefore, the need exists for alternative methods to provide a natural and smooth appearance to the skin with visible reduction in wrinkles, fine lines, pores and skin imperfections but which overcomes the problems associated with previous methods and compositions and which would represent a significant advance in cosmetic art.

The optical reduction of wrinkles is due to the light diffusing properties of the applied particles. At the margins and in the creases of wrinkles, particles that scatter and thus diffuse light away minimize the depressions in the skin. To the observer, the wrinkles appear blurred, hence the terms “soft focus effect” “blurring effect.” In the past, the blurring effect was based on the diffuse reflection of spherical particles such as microspheres and fibers. One such composition is that described by Nakamura, N. et al, “Blurring of Wrinkles Through Control of Optical Properties”, XIVth I.F.S.C.C. Congress, Barcelona, Spain, 1986.

The incorporation of inorganic nanoscale particles into a polymeric matrix is known for industrial uses to provide clear coatings, for example, mobile phones or skies.

### SUMMARY OF THE INVENTION

The present invention provides for the use of fractal particles with unique optical properties and surface chemistry, combined with micron dimension organic or inorganic particles such that the fractal network wrap around the macroscopic particles increasing the interfacial area over which lateral light diffusion occurs. It has been found that such technology is useful to optimize the optical diffusion effect of light, i.e., optical blurring, and consequently, cause the appearance of wrinkles, fine lines, pores and skin imperfections to vanish while allowing the skin to appear natural and flawless.

It is an object of the present invention to provide a gel system (as hereinafter further described) comprising macroparticles within a fractal network of nanoparticles.

Another object of the present invention is to provide such gel system comprising macroparticles that are translucent, for example silicone crosspolymers.

A further object of the invention is to provide the gel system in which the fractal network is a fractal gel (as hereinafter further described).

It is yet another object of the present invention to provide cosmetic compositions comprising the gels of the present invention that are efficient in blurring fine lines, wrinkles, pores, and skin imperfections.

It is a further object of the present invention to provide gels that leverage the differences in size domain and optical properties between fractal particles and macroscopic particles. The presence of macroscopic particles increase the spatial distribution of fractal particles increasing the interfacial area over which light bending/lateral scattering occurs. Accordingly, the gels are seen to have superior optical properties when used especially in cosmetic products. Macroscopic particles can be organic or inorganic. Non-limiting examples of macroscopic particles are silicone elastomers, hydrocarbon elastomers, silicone crosspolymers, polymeric spheres, metal oxide spheres, or combinations thereof.

It is a further object of the present invention to provide cosmetic compositions containing aqueous gels according to the invention comprising macroscopic particles present in a fractal particle network obtained by using a mixture of fractal particles with opposite zeta potential at a given pH. Such aqueous gels may be used as prepared, may be modified to include other ingredients, or may be incorporated as a phase in an emulsion cosmetic composition. Preferred macroscopic particles in the present invention are silicone elastomers and silicone crosspolymers.

It is another object of the present invention to provide cosmetic compositions containing anhydrous gels of the invention comprising macroscopic particles dispersed in a network of fractal particles, typically with a compatible anhydrous solvent. Such anhydrous gels may be used as prepared, may be modified to include other ingredients, or may be incorporated as a phase in an emulsion cosmetic composition.

A further object is to provide methods for producing the gels of the invention and cosmetic compositions containing same.

The invention also has as its object a cosmetic treatment process allowing wrinkles, fine lines, pores and skin imperfections to be blurred in human beings, particularly the skin of the face, neck, and lips, this process being characterized by applying an effective quantity of a composition of the present invention to the skin.

Further according to this and other objects and advantages of the present invention, there are provided methods for blurring wrinkles and fine lines. A method includes applying to the skin and/or lips a gel composition which leverages the relative size/domains and refractive indices of the fractal network and macroscopic particles to obtain efficient blurring.

In another aspect of the invention the present invention is applicable to the skin in a cosmetically acceptable vehicle.

These novel features of the present invention will become apparent to those skilled in the art from the following detailed description, which is simply, by way of illustration, various modes contemplated for carrying out the invention. As will be realized, the invention is capable of additional, different obvious aspects, all without departing from the invention. Accordingly, the Figures and specification are illustrative in nature and not restrictive.

### BRIEF DESCRIPTION OF THE FIGURES

FIG. 1 is a schematic representation of the spatial arrangement of the gel structure comprising the macroparticles and the fractal nanoparticles.

FIG. 2 is a schematic representation illustrating the diffusion on light incident on the surface of skin treated with a cosmetic composition of the present invention.

FIG. 3 is a graphical plot of the zeta potential of various metal oxides as a function of pH.

### DETAILED DESCRIPTION OF THE INVENTION

The present invention utilizes gel systems to provide compositions having unique optical and space filling properties. As a consequence to the optical properties, thin films of the compositions applied to a substrate, in particular, a biologic surface, change the way light incident on the surface of the film is refracted and improves the diffusion of incident light on the surface of the film. When the composition is a cosmetic composition and the film is on the surface of the skin of an individual, the imperfections of the skin are less noticeable, i.e., less “visible” because of the way reflected light is being seen by an observer. In addition the gels of the present invention provide a fractal network with macroscopic particles, thus further providing beneficial optical properties, especially when the refractive indices are non-matching. The gels are capable of filling voids in the surface of the biologic substrate, especially, wrinkles, lines, pores, and other imperfections present in the surface of an individual's skin or lips. Unless indicated otherwise, the term “gel” refers to the gels as set forth in this paragraph and as further described in this specification.

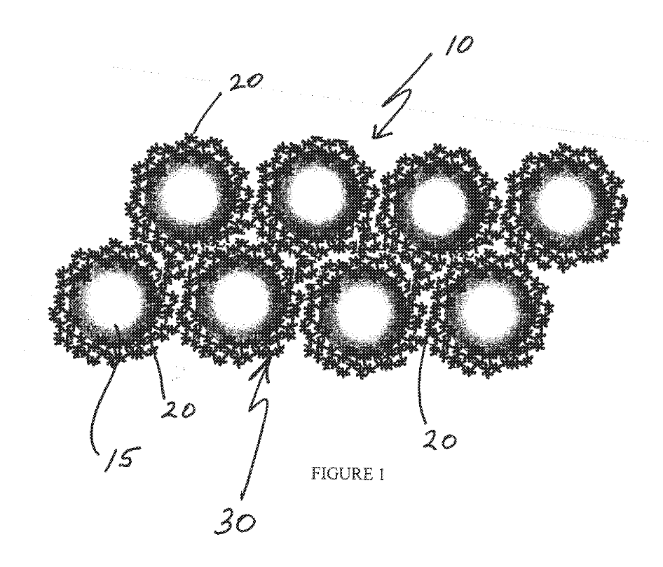

As shown schematically in FIG. 1, it is believed that gel 10 comprises a plurality of translucent macroparticles 15 surrounded by a multiplicity of nanoparticles 20, which nanoparticles aggregate or otherwise coalesce to form a fractal gel network, represented generally by numeral 30.

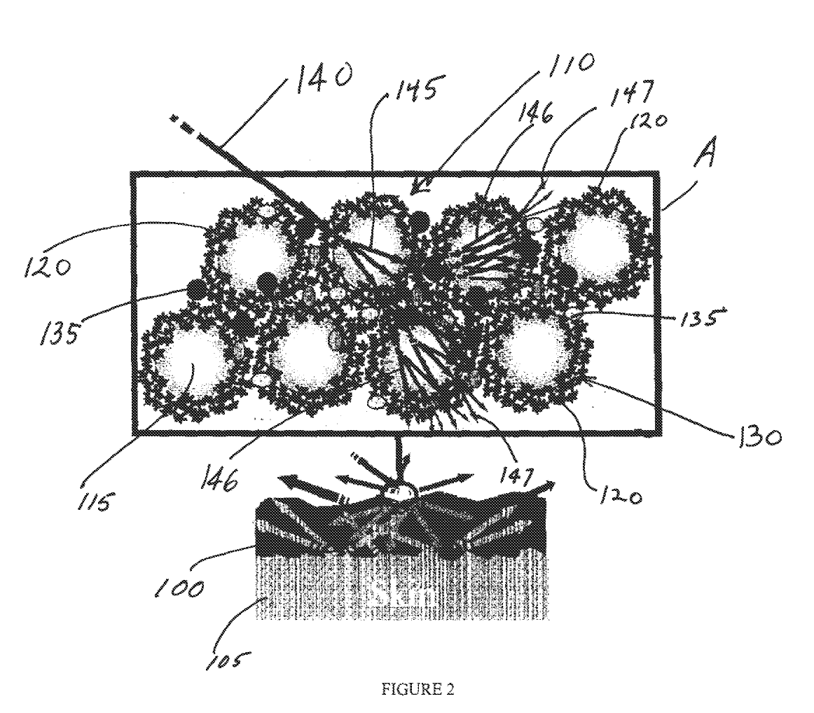

FIG. 2 illustrates a film 100 of a cosmetic composition of the present invention applied to skin 105, as well as an enlarged view A of the gel 110 taken from a small area of the film 100. Gel 110 comprises a plurality of translucent macroparticles 115 surrounded by a multiplicity of nanoparticles 120, whereby fractal gel network 130 is formed. Actives and/or adjuvants 135 are present within the gel network 130. Light 140 entering the gel 110 is diffused by the translucent macroparticles, as shown schematically by the plurality of light vectors 145, 146, and 147, whereby the skin is provided with an optical blurring benefit.

Another beneficial aspect of the invention is the ability of the gel network to display unique rheological properties, which are especially useful in cosmetic applications. The gel network is highly thixotropic. That is to say, the viscosity of the gel rapidly diminishes under increasing shear stress, yet the gel network reforms quickly once the shear stress is removed. Effectively, this imparts an effect wherein the composition transforms from viscous, non-flowing compositions to a free flowing liquid when the composition is applied, e.g., with a brush or other applicator. The speed at which the network reforms to a gel is a function of particle concentration, and, in the instance where the fractal network is a fractal gel, on the magnitude of the attractive interaction between the oppositely charged particles (refer to section “Surface Charge of Particulate Dispersions”). Hyperthixotropic compositions are particularly useful in foundations, mascaras, hair care, lip compositions, and personal care compositions where low viscosity is desired during application, yet a rapid increase in viscosity is important to prevent migration of the applied composition.

The gel system of the present invention comprises one or more translucent macroparticles and includes a fractal network of nanoparticles. Translucent materials allow light to pass through them but scatter light so that the material distorts the image. Suitably translucent macroparticles are those whose diffuse transmittance is greater than zero for a 10 micron film cast on a glass plate as measured using a color(i) spectrophotometer. Films can be prepared by dispersing macroparticles in a suitable binder, polymer, or solvent. A dispersion can prepared by dispersing macroparticles in a binder, polymer or solvent followed by casting a 10 micron film on a glass (normalized with % solids in the solution) using a drawdown bar. A color (i) spectrophotometer can be used to measure total transmittance and direct transmittance. Diffused transmittance can be obtained by subtracting direct transmittance from total transmittance.

In the present invention the fractal network comprises one or more types of fractal particles. In the preferred embodiment the fractal network is a fractal gel. While not wishing to be bound by any particular theory or mechanism, the fractal network is believed to envelop the macroparticles, with gelation occurring when dispersed in a suitable medium.

The phrase “cosmetically acceptable vehicle” refers to a medium that is compatible with keratin materials, such as human skin.

The term “optical blurring” as used herein refers to optical reduction of wrinkles, fine lines, skin surface unevenness and imperfections.

The term “macroscopic particles” or “macroparticles” as used herein refers to particles that have a size range of 1 to 200 microns.

The term “nanoparticle” refers to particles with a size of up to about 900 nm.

The term “fractal particles” as used herein refers to nanoparticles of varying fractal dimension or in-built reticulated structure; that is, having Hausdorff-Besicovitch dimensions greater than their topological dimensions. As used herein, “nanoparticles” is synonymous with “fractal particles”, unless specifically indicated otherwise.

By “gel” is meant any co-continuous phases of macroscopic particles and a fractal particle network that forms a composite gel structure.

The terms “blurring” and “optical blurring” as used herein refers to optical reduction of wrinkles, fine lines, pores and skin surface unevenness and imperfections.

Reference to “particle size” means the mean diameter of particles measured under an appropriate imaging technique for the size domain under consideration, for example, scanning electron microscopy or transmission electron microscopy.

Except where specific examples of actual measured values are presented, numerical values referred to herein should be considered to be qualified

The terms “a” and “an”, as used herein and in the appended claims, mean “one or more” unless otherwise indicated herein.

All percentages and ratios referred to herein are by weight of total composition (i.e., the sum of all components present), unless otherwise indicated.

### The Fractal Network

In one embodiment the fractal network comprises at least one type of submicron sized fractal particle (i.e., nanoparticles). The fractal particles may have a positive, neutral or negative net surface charge (zeta potential. When dispersed in a suitable medium the fractal particles coalesce to form the fractal network. Depending on the medium, the coalescing takes place in light of van der Waals forces (hydrophilic dispersant), electrostatic attraction, or because of hydrogen bonding (lipophilic dispersant). As explained in more detail below, the macroparticles may be added to this dispersion under shear to form the gels of the present invention.

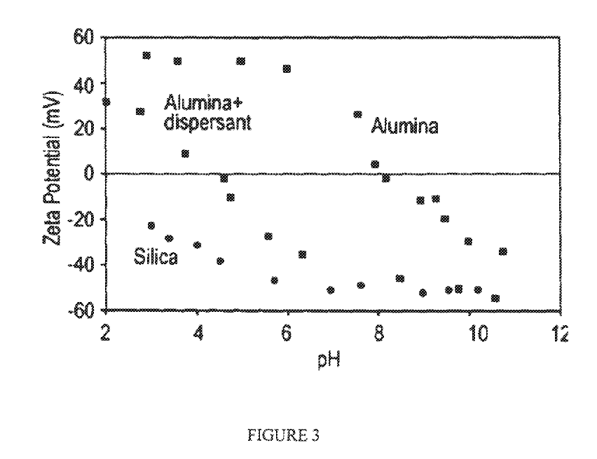

In one embodiment the fractal network is a fractal gel. The fractal gel comprises first and second submicron sized fractal particles having opposite surface charges at a given pH. By way of example and referring to FIG. 3, at pH below 7-8 the metal oxides silica and alumina have opposite surface charge or zeta potential. The first or second fractal particles that form the fractal gel may each comprise two or more different fractal particles having the same charge. The two or more different first or second fractal particles of the same charge may have different sizes, different net surface charges (of the same type, however), or different refractive indices. The use of fractal gels in the formation of gels of the present invention is preferred. The fractal gels, because of their oppositely charged particles, have stronger attraction between the fractal particles.

The fractal gel is obtainable by forming dispersions of the oppositely charged fractal particles. Combining aqueous dispersions of each particle type forms a highly structured gel network as a result of charge neutralization. Alternatively, the zeta potentials may be of the same sign initially, and the pH of the combined dispersions adjusted thereafter to give zeta potentials of opposite signs, thereby allowing integration of the organic particles and inorganic particles.

When the fractal network comprises a single fractal particle or two or more fractal particles of the same or neutral charge (i.e., not oppositely charged as needed to form the fractal gels), the network can be formed by providing a dispersion of the fractal particles as described in the previous paragraph. A network will form suitable for use in the present invention when the cohesive interactions among particles is greater than adhesive interactions between the particles and the medium. The macroscopic particles are incorporated following network formation.

A brief description of fractal particle geometry follows:

Fractal objects are characterized by a recursive self-similarity. In general, the fractal nature can be described mathematically by a power law relationship taking the form:

Y=c*X^{d} (1)

where c is a constant. Therefore, if data adhere to a power law relationship, a plot of log(Y) versus log(X) will yield a straight line with slope d.

Analogously, self-similar fractals, a class of Hausdorff-Besicovitch dimensionality, rely on the object being self-similar at different length scales. The power law is consistent with this case following:

A=(1/s)^{D} (2)

where A is the number of identical parts, s is the reduction factor and D is the self-similar dimension measure of the fractal. Equation 2 can be arranged as the following

D=log(A)/Log(1/s) (3)

For example, the sides of a unit square are divided in half, forming 4 pieces, therefore A=4, s=½ thus D equals 2. Likewise a Sierpinski Gasket, wherein the original triangle side is halved, three triangle pieces are formed. Thus, A=3, s=½ and D=1.5850. Comparatively, consider a unit line segment. Dividing the line in half results in 2 equal parts, and so on. Therefore, A=2, s=½ D=1. It is important to note, the value of D agrees with the topological dimension of the line, yet a line is not fractal. Accordingly, fractals are those objects wherein the Hausdorff-Besicovitch dimension exceeds its topological dimension.

Furthermore, fractals can be classified according to their self-similarity. There are three basic types of self-similarity expressed in fractals. Exact self-similarity (the strongest type of self-similarity). The fractal appears identical at different length scales. Fractals of this type are described by displaying exact self-similarity.

Quasi-self-similarity (non-exact form of self-similarity). The fractal appears approximately identical at different length scales. Quasi-self-similar fractals are comprised of distorted and degenerate copies.

Statistical self-similarity (weakest type of self-similarity). The fractal is described by statistical measures, which are preserved across the length scale. Random fractals are examples of fractals, which are statistically self-similar, but not exact or quasi self-similar. The nature of similarity of fractals can also be described by mathematical functions.

Most fractal objects of interest do not have a readily apparent self-similar nature. Therefore, a convenient method to determine the fractal dimension of the object is the box counting method. This method is widely used and a direct method to measure the fractal dimension objects via image analysis. An object image is projected on a grid of known dimensions. Subsequently, the number of blocks that the image touches is counted. This data yields the number of blocks (N) and the block size (reduction factor, s). The grid is resized, and the process is repeated. A plot of the data, where the x-axis is log(s) and the y-axis is log(N(s)) using equation 3, yields a slope of value D.

Image analysis is particularly useful to evaluate the fractal dimension of particulates. Specifically, transmission electron spectroscopy (TEM) is well adapted to evaluate the fractal dimension of complex particulate structures. Of particular interest are fractal particles that are comprised of non-overlapping primary particles, which form a larger aggregate structure. Typically, particles of this nature are manufactured by a fuming process or complex precipitation process.

Evaluation of the mass fractal dimension of particles formed from aggregates of smaller primary particles involves determination of the number of primary particles per aggregate. Typically, this is achieved by evaluating TEM micrographs using digital imaging processing techniques. The number of primary particles per aggregate (N) is determined by dividing the projected area of the aggregate (Aa) by the projected area of the monomer unit (Am):

N=(Aa/Am)^{α} (4)

where α is an empirical fitting parameter, typically 1.0-1.1. Therefore, the Hausdorff dimension implies the relationship between the primary particle size (dp), the area radius of gyration (Rg), and the number of primary particles (N) describes the fractal dimension (Df) of the aggregate:

N=kf(Rg/dp)^{Df} (5)

where kf is a constant fractal prefactor. A plot of log(N) vs. log(Rg) results in a linear plot of slope Df. Typical Df values for fractal particles of the present invention obtained by a fuming process range from 1.5-1.9, while fractal particles of the present invention obtained by a precipitation process range from 2-2.8.

Additional test methods based on rheological measurements and light scattering measurements can be used to elucidate the dimensionality of fractal particles, as known in the art.

The fractal nature of the particles results in a porous matrix structure of the fractal network. In another embodiment the porous matrix structure of the fractal network may receive one or more active substances.

The size domains and refractive indices of the fractal particles are chosen to effectively form a barrier between the macroscopic particles and consequently enhance the ability of the compositions of the invention to fill wrinkles and mask skin imperfections. The fractal particle network has an open structure, which provides a surface smoothing effect. Thus, the composition fills in imperfections in the surface of the skin, and thus provides a natural, smooth and youthful appearance with visible reduction in wrinkles and skin imperfections when applied to the skin.

Typically, the fractal particle may comprise between about 5% to about 75%, preferably about 10-40%, most preferably about 20-40% solid fractal particles by weight of the fractal particle dispersion. In some instances the particles are provided by the manufacturer as a dispersion. Suitable commercially available metal oxide dispersions are Cab-o-Sperse™ PG01, PG063, PG003, PG0042, and AeroDisp™ W1836, W630 supplied by Cabot Corporation and Degussa, respectively.

It is also possible to provide nonaqueous dispersions that can be used to form a nonaqueous gel phase. Where required, the dispersion media must be able to maintain the surface charge of the fractal particle, typically requiring trace quantities of a charge control agent such as tetrabutyl ammonium benzoate, so that charge neutralization may occur. Suitable dispersion media that may be used to provide the dispersion of the fractal particles are hydrocarbons such as isododecane, simple esters, and silicone fluids such as cyclomethicone. Ionization of metal oxide surface in nonaqueous media is discussed in: Labib, M. E.; Williams, R. J.; J. Colloid Interface Sci. 1984, 97, 356; Labib, M. E.; Williams, R. J.; J. Colloid Interface Sci. 1987, 115, 330; Fowkes, et al., “Mechanism of Electric Charging of Particles In Nonaqueous Dispersions”, Journal of the American Chemical Society, vol. 15, 1982; Fowkes, et al., “Steric And Electrostatic Contributions To The Colloidal Properties of Nonaqueous Dispersions”, Journal of the American Chemical Society, vol. 21, 1984; Huang, Y. C., Sanders, N. D., Fowkes, F. M., Lloyd, T. B. “The Impact of Surface Chemistry on Particle Electrostatic Charging and Viscoelasticity of Precipitated Calcium Carbonate Slurries”. National Institute of Standards and Technology Special Publication 856, USA Department of Commerce, 180-200 (1993)).

Any suitable metal oxide fractal particles or derivatives thereof that achieve result in a reticulated fractal network may be employed. Preferably, the inorganic nanoparticles particles are fractal metal oxide particles having a diameter of between about 25-300 nm, preferably about 40-250 nm, and more preferably about 40-200 nm. Diameter as used herein refers to the diameter of a sphere that encompasses the fractal particle. Diameter may be determined by methods known in the art, e.g., light scattering and TEM. Furthermore, each nanoparticle type has a particle surface area between about 50 to 400 m²/g, and more particularly between about 50 to 300 m²/g. The fractal dimension of the nanoparticle ranges from about 1.2 to 2.8, preferably from about 1.5 to 2.5. Generally, as fractal dimension increases, the concentration of solids in the network decreases, and as surface area increases, fractal dimension also increases.

While not common, fractal organic particles are known and can be used in accordance with the present invention, provided the requisite surface charge characteristics are met. For example, organic polyacrylates and their derivatives have fractal dimensionality and may be surface charged. Preferred organic polyacrylate particles are lauryl methacrylate/dimethyl acrylate crosspolymer (available from Amcol Health and Beauty Solutions).

The fractal particles may be selected from the group consisting of silica, alumina, titania, zirconia, zinc oxide, indium tin oxide, ceria, and mixtures thereof. Particles may be formed as part of a fuming process or a precipitation process wherein the metal oxide particle is fractal in dimension. Particles formed by the fuming process are preferred. Alumina is known to impart high diffuse transmittance, high reflectance, high scattered reflectance and low total reflectance in the visual spectra, and is a preferred fractal particle. Silica is preferred because it has a refractive index that is substantially matchable to common cosmetic media, especially silicone oils.

When the fractal network is a fractal gel, alumina and silica are preferred oppositely charged particles. As shown in FIG. 2, silica is available with a net surface charge that is opposite to that of alumina at a pH value of most cosmetic formulations, that is, at a pH below about 7-8. Accordingly, silica is a preferred second fractal particle, especially when used in conjunction with alumina at a composition pH less than about 7 to 8.

Examples of suitable fractal particles include, but are not limited to, fumed silicas sold by Degussa under the tradename Aerosil, including hydrophilic and hydrophobic fumed silicas, for example, the Aerosil R-900 series, A380™ fumed silica (manufactured by Degussa), OX50™ (manufactured by Degussa), colloidal silica such as the Cabosil™ line (manufactured by Cabot), fumed alumina such as SpectrAL™ (manufactured by Cabot), and fumed titania. Preferred is fumed silica, fumed alumina, fumed titania (Degussa W740X) fumed zirconia (Degussa W2650X, W2550X), fumed ceria (Degussa Adnano) fumed zinc oxide (Degussa Adnano), fumed indium tin oxide (Degussa Adnano) or mixtures thereof.

Charged particles are subject to electrophoresis, that is to say, in the presence of an electric field they move with respect to the liquid medium in which they are dispersed. The region between the particle and the liquid is known as the plane of shear. The electric potential at the plane of shear is called the zeta potential. The magnitude and sign of this potential can be experimentally determined using commercially available equipment. Typically, to achieve colloidal stability, i.e. prevent flocculation, charged particulates are required to have a minimum zeta potential of approximately 25 mV.

Selection of fractal particle pairs for the fractal gel can be chosen based on the magnitude and sign (positive or negative) of the zeta potential at a given pH. Preferably, the magnitude and sign of the zeta potential of each particle type is sufficient, such that when combined, a non-settling, semi-rigid gel structure is formed. Preferred dispersions of the first particle type have a zeta potential values of about +10 mV to +50 mV, more preferably +10 mV to +30 mV, and most preferably +15 mV to +25 mV. Preferred dispersions of the second particle type have a zeta potential values of about −10 mV to −50 mV, more preferably −10 mV to −30 mV, and most preferably −15 mV to −25 mV. Furthermore, evaluation of the point of zero charge (isoelectric point) of metal oxides is useful to pre-select metal oxides of interest, as listed in Table 1.

### Surface Charge of Particulate Dispersions

The presence of charge on dispersed colloidal particles occurs by two principal mechanisms: dissociations of ionogenic surface groups or preferential absorption. Each mechanism can occur simultaneously or independently. Dissociation of acidic groups on the surface of a particle will give rise to a negatively charged surface. Conversely, dissociation of basic surface groups will result in a positively charged surface. In both cases, the magnitude of the surface charge depends on the strength of the acidic or basic groups and on the pH of the solution. The surface charge can be reduced to zero (isoelectric point) by suppressing the surface ionization. This can be achieved by decreasing the pH in the case of negatively charged particles or increased the pH in the case of positively charged particles. Furthermore, if alkali is added to a dispersion of negatively charged particles, the particles tend to become more negatively charged. If acid is added to this dispersion, then a point will be reached where the charge on the particle is neutralized. Subsequent addition of acid will cause a build up of positive charge on the particle.

### Modification of Surface Charge

Adsorption of ions and ionic surfactants can be specifically adsorbed onto the charged particle surface. In the case of cationic surfactants, adsorption leads to a positively charged surface and in the case of anionic surfactants, adsorption leads to a negatively charged surface. Adsorption of single valent or multivalent inorganic ions (e.g. Na⁺, Al⁺³) can interact with charged surfaces in one of two ways: reduction of the magnitude of charge at a given pH; change in pH of the isoelectric point (point of neutral charge). The specific adsorption of ions onto a particle surface, even at low concentrations, can have a dramatic effect on the surface charge. In some cases, specific ion adsorption can lead to a charge reversal of the surface. The addition of surfactants or specific ions to particle dispersions is a common method to modify the surface charge characteristics.

TABLE 1
Point of Zero Charge (PZC) for
Various Oxides in Water

| Oxide | PZC |
|---|---|
| Ag₂O | 11.2 |
| Al₂0₃ | 9.1 |
| BeO | 10.2 |
| CdO | 11.6 |
| CeO₂ | 8.1 |
| CoO | 10.2 |
| CO₃O₄ | 7.4 |
| Cr₂O₃ | 7.1 |
| CuO | 9.3 |
| Fe₂O₃ | 8.2 |
| Fe₃O₄ | 6.6 |
| HgO | 7.3 |
| La₂O₃ | 10.1 |
| MgO | 12.4 |
| MnO₂ | 5.3 |
| MoO₃ | 2 |
| Nb₂O₅ | 2.8 |
| NiO | 10.2 |
| PuO₂ | 5.3 |
| RuO₂ | 9 |
| Sb₂O₅ | 1.9 |
| SiO₂ | 2 |
| SnO2 | 5.6 |
| Ta₂O₅ | 2.8 |
| ThO₂ | 9.2 |
| TiO₂Rutile | 5.7 |
| TiO₂Anatase | 6.2 |
| V₂O₃ | 8.4 |
| WO₃ | 0.4 |
| Y₂O₃ | 8.9 |
| ZnO | 9.2 |
| ZrO₂ | 7.6 |

In one embodiment the fractal network is obtained by using at least one kind of fractal particle. The fractal particles may be of the same charge and are not limited by a particular pH.

### The Macroscopic Particle

The macroscopic particles used in the preparation of the gels of the present invention are translucent, and are within the matrix of the gel. However, it is understood that the cosmetic composition may be multiphasic, for example, an emulsion wherein the gel is dispersed into a continuous external phase. Alternatively, additional components may be dispersed into the gel phase. In yet another embodiment, macroscopic particles may have a charge or surface functionality which increases interaction with the fractal particles.

In yet another embodiment, two different fractal particles are used to form different fractal networks, which are used to form, along with macroscopic particles, the gels of the invention. In another embodiment different macroscopic particles can be used to form the gel, using the same or different fractal networks.

Preferably, the refractive index of the fractal particle does not match the refractive index of the macroparticle. Non-match means as used in this context that the refractive index values are about 0.05 or more units from one another, preferably more than about 0.07, and most preferably more than about 0.1.

The macroscopic particles of the invention have a particle size of between about 1-200 microns, preferably between about 2-100 microns. Macroscopic particles can be organic or inorganic. Non-limiting examples of macroscopic particles are silicone elastomers, hydrocarbon elastomers, silicone crosspolymers, polymeric spheres, metal oxide spheres, or combinations thereof. In one preferred embodiment of the invention, the macroscopic particles are macroscopic organic elastomeric particles. In another preferred embodiment the macroscopic particles are silicone crosspolymers having a particle size of from about 1 to about 200 microns.

Illustrative, non-limiting examples of elastomeric macroparticles to which this invention may be applied are natural and synthetic rubbers, for example, natural rubber, nitrile rubbers, hydrogenated nitrile rubbers, ethylene-propylene rubbers, polybutadiene, polyisobutylene, butyl rubber, halogenated butyl rubber, polymers of substituted butadienes. such as chlorobutadiene and isoprene, copolymers of vinyl acetate and ethylene terpolymers of ethylene, propylene, and a non-conjugated diene, and copolymers of butadiene with one or more polymerizable ethylenically unsaturated monomers such as styrene, acrylonitrile, and methyl methacrylate; silicone elastomers; fluoropolymers including fluoropolymers having a silicone backbone; polyacrylates; polyesters, polyacrylic esters, polyethers; polyamides, polyesteramides, polyurethanes, and mixtures thereof. Moreover, it is understood that the elastomeric material may contain additional organic or inorganic phases to modify the elastomeric and optical properties of the particle.

Such particles are prepared by conventional procedures, for example, by palletizing, cutting, or tearing a bale of the elastomeric material into shreds or small pieces followed by chopping or grinding those shreds or small pieces into particles having the size desired. In addition “wet” chemistry techniques known in the art may be used to form particles of a particular size or distribution of particle sizes that are desirable. The practice of the present invention does not depend on the particular procedure utilized to prepare the elastomer and elastomeric particles.

Suitable macroscopic particles useful in the invention especially for skin care applications have a preferred refractive index generally from about 1.38 to about 2, preferably from about 1.38 to about 1.6.

The silicone elastomers are (i) cross-linked silicone polymers derived from room temperature vulcanizable silicone sealant chemistry, or (ii) addition polymerized silicone elastomers prepared by the hydrosilylation of olefins or olefinic silicones with silyl hydrides. Preferred silicone elastomers are crosslinked organopolysiloxanes such as dimethicone/vinyl dimethicone crosspolymers, vinyl dimethicone/lauryl dimethicone crosspolymers, alkyl ceteayl dimethicone/polycyclohexane oxide crosspolymers, and mixtures thereof. Examples of these elastomers are DC 9040, DC 9040, and DC 9045 available from Dow Corning, dimethicone/phenyl vinyl dimethicone crosspolymers under the tradenames KSG-15, 16, 18 available from Shin Etsu; lauryl dimethicone/vinyl dimethicone crosspolymers supplied by Shin Etsu (e.g., KSG-31, KSG-32, KSG-41, KSG-42, KSG-43, and KSG-44), and the Gransil line of elastomers available from Grant Industries such as EPSQ™. A preferred silicone elastomer is EPSQ™ (Grant).

Also suitable are silicone crosspolymers obtained by self polymerization of bifunctional precursor molecules containing both epoxy-silicone and silyl hydride functionalities to provide a silicone copolymer network in the absence of crosslinker molecules. Such crosspolymers are described in U.S. Pat. Nos. 6,444,745; 6,531,540; 6,538,061; 6,759,479, and in US Appln. 2003/00959935. Especially suitable are such crosspolymers such as the Velvesil™ line of silicone crosspolymers available from Momentive Performance Materials, Inc. (formerly GE Silicones). Most preferred is Velvesil 125™ (GE), a cyclomethicone and C30-C45 alkyl cetearyl dimethicone/polycyclohexane oxide crosspolymer.

The weight ratio of the fractal particles to macroscopic particles in the gels of the present invention are typically from about 1:10 to 10:1, preferably from about 1:10 to 2:1, and most preferably from about 1:5 to 1:1.

Other preferred macroscopic particles are polymeric spheres such as nylon (e.g., Nylon 12 available from Cabot as SP-500 and Orgasol 2002™), cellulose beads, poly(methyl acrylic acid) (also known as PMMA or methyl methacrylate cross polymer; CAS No. 25777-71-3), boron nitride, barium sulfate, silicates such as calcium alumina borosilicate, polyethylene, polystyrene, polyurethane such as HDI/Trimethylhexyl lactone cross polymer sold by Kobo Industries under the tradename BPD-800, ethylene/acrylic acid copolymer (e.g. EA-209 supplied by Kobo), Teflon, or silicone.

### Compositions of the Invention

The cosmetic composition may take on various forms including powder, cake, pencil, stick, ointment, cream, milk, lotion, liquid-phase, gel, emulsion, emulsified gel, mousse, foam, spray, wipes. Preferably, the cosmetic composition is used in a liquid or powder foundation.

The gels may be incorporated in cosmetically acceptable vehicles, such as but not limited to, liquid (e.g., suspension or solution), gel, emulsion, emulsified gel, mousse, cream, ointment, paste, serum, milk, foam, balm, aerosol, liposomes, solid (e.g., pressed powders), anhydrous oil and wax composition. Preferably, the cosmetic composition is used in a liquid or powder foundation. More specifically, the cosmetic include facial skin care cosmetics such as skin lotion, skin milk, skin cream, gel, and make-ups such as foundation, foundation primer base, blush, lip stick, eye shadow, eye liner, nail enamel, concealer, mascara, body make-up product, or a sunscreen.

A person skilled in the art can select the appropriate presentation form, and also the method of preparing it, on the basis of general knowledge, taking into account the nature of the constituents used and the intended use of the composition.

Cosmetic compositions according to the invention may comprise from about 1 to 100% gel, and typically comprise from about 2 to about 90%, preferably comprise 10 to 80%, and most preferably comprise 30 to 55% gel by weight of the cosmetic composition. The broad range reflects the range of different types of cosmetic products and the various product forms; namely, gels, emulsions, and dispersions. Typically, the gel contains about 3% to about 60% fractal particles by weight of the gel, and more typically from about 5% to about 40% fractal particles by weight of the gel. Amounts of the gel in the cosmetic compositions of the invention are also discussed later. Useful gel compositions may include alumina and silica, titania and silica, zirconia and silica, and other combinations of particulates described within.

The cosmetic compositions of the present invention may be formulated as aqueous or nonaqueous compositions, which may be emulsions or non-emulsions. In one embodiment, the cosmetic compositions according to the invention are formulated as emulsions. These emulsions may be oil-in-water (including silicone in water) emulsions, water-in-oil (including water-in-silicone) emulsions, or multiple emulsions such as oil-in-water-in-oil (o/w/o) or water-in-oil-in-water (w/o/w), but are preferably silicone-in-water emulsions. It is understood that the oil phase can comprise silicone oils, non-silicone organic oils, or mixtures thereof. While not preferred, the compositions can comprise two immiscible phases that are admixed at the time of use by shaking.

In a typical embodiment in which a gel having a fractal gel is employed, the weight ratio of alumina to silica is 1:1 to 9:1 and is present as a dispersion in water wherein the alumina surface area is between 50 to 200 m2/g and the silica surface area is between about 50 to 400 m2/g. Suitable gels can be formed by using Spectral 51 or Spectral 80 (Cabot Corporation) fumed alumina and Cab-O-Sil M5, Cab-O-Sil EH-5. Furthermore, dispersions of metal oxides can be chosen based on their surface charge characteristics as determined by zeta potential measurements. Generally, particle size, surface area and surface charge determines the ease with which the gel forms and its physical attributes such as yield strength. In one embodiment of the invention the gel comprises, as the only fractal particle, a fumed silica or a fumed alumina,

In addition to the gel phase comprising the fractal particles and the macroparticles, the compositions of the present invention may comprise one or more active ingredients adapted to bestow a cosmetic benefit to the skin when applied to the skin as a film and/or one or more adjuvants or excipients (adjuvants and excipients are collectively referred to herein as adjuvants) to impart to the cosmetic product particular desirable physical properties, to meet product performance requirements, or to establish compositional type, e.g., emulsion (of a particular type), solution, etc. The actives and/or the adjuvants may be present in the gel phase including the fractal network or the fractal gel, as the case may be, in another phase, or in either, as desired, or as mandated by the chemical system.

Suitable active agents include pigments to impart a color to the skin or other biologic surface; opacifiers and light diffusers; sunscreens; uv light absorbers; emollients; humectants; occlusive agents; antioxidants; exfoliants; antioxidants; anti-inflammatory agents; skin whitening agents; abrasives; antiacne agents; hair treatment agents; humectants; emollients; moisturizers; anti-wrinkle ingredients; concealers; matte finishing agents; proteins; anti-oxidants; bronzers; solvents; ultraviolet (UVA and/or UVB) absorbing agents; oil absorbing agents; neutralizing agents. It is understood to those skilled in the art that any other cosmetically acceptable ingredient, i.e., those included in the International Cosmetic Ingredient Dictionary and Handbook, 11th Edition (2006) (Cosmetic and Toiletries Association) (hereinafter identified as INCI) may be used and compatible combinations thereof.

Suitable adjuvants include film forming agents; solvents; viscosity and rheology modifiers such as thickeners; surface active agents including emulsifiers; hydrotropes; emulsion stabilizers; plasticizers; fillers and bulking agents; pH adjusting agents including buffers, acids, and bases; chelating agents; binders; propellants; fragrances; preservatives and antimicrobials, and compatible combinations thereof.

Suitable active agents and adjuvants used in cosmetic compositions of the present invention are tabulated in INCI. Generally, reference to specific materials utilizes the INCI adopted name nomenclature. The active agents and adjuvants are incorporated in the compositions of the present invention in amounts that provide their intended functions, as those skilled in the cosmetic arts are knowledgeable. Generally, this amount is from about 0.001 to 25%, more usually 0.01 to 15%, and especially 0.1 to 10% by weight of the composition.

The cosmetic compositions may contain polymeric light diffusers such as nylon (e.g., Nylon 12 available from Cabot as SP-500 and Orgasol 2002™), cellulose beads, poly(methylacrylic acid) (also known as PMMA or methyl methacrylate crosspolymer; CAS No. 25777-71-3), polyethylene, polystyrene, ethylene/acrylic acid copolymer (e.g., EA-209 supplied by Kobo), and fluorinated hydrocarbons such as Teflon. The polymeric light diffusers, preferably nylon, are present in a concentration in the range of between about 0.01-10%, preferably about 0.1-5% by weight of the composition. Inorganic light diffusers can also be used, e.g., boron nitride, barium sulfate, and silicates such as calcium alumina borosilicate, and are typically present in an amount of from about 0.01 to about 10%, preferably about 0.1 to about 5% by weight.

The cosmetic composition of the present invention may contain a viscosity modifier such as a thickener together with emulsifiers to modify the viscosity of the composition, for example to form creams, pastes, and lotions that enhance skin feel. Suitable viscosity modifiers are starches, cellulose derivatives such as sodium carboxymethyl cellulose, methyl cellulose, ethyl cellulose, cationized cellulose, hydroxyethyl cellulose, hydroxypropyl cellulose, hydroxypropylmethyl cellulose; silicates such as Veegum or clays; polysaccharides such as xanthan or guar gums, hydrophilic polymers, such as carboxyvinyl polymers, for example carbomers. Viscosity/rheology modifiers may be present in the composition in an amount of from about 0.1 to about 10% by weight of the composition.

The cosmetic emulsifier should preferably be an oil-in-water or water-in-oil emulsifier. Preferably, the oil phase is a silicone oil, and the emulsifier is a silicone emulsifier. The emulsifiers may be chosen advantageously to match the refractive index of the fractal particle whose refractive index is matched, but are not substitutes for the refractive index matching polymer.

Emulsifying agents may be present in a concentration of from about 0-10%, preferably about 0.1-6%, more preferably about 3-5%. Nonlimiting examples of suitable emulsifiers are glycerol monostearate, PEG 12 Dimethicone (Dow Corning), RM 2-2051™ (Dow Corning), an emulsion of aqueous polyacrylate emulsified into silicone (dimethicone and cyclopentasiloxane), alkylmethyl siloxanes copolyol (Dow Corning 5200), PEG 11 methylether dimethicone (Shin Etsu), cyclopentasiloxane/PEG/PPG 18/18 dimethicone (Dow Corning 5225C).

The cosmetic composition of the present invention may contain non-occlusive film-forming agents such as, but not limited to, cosmetic fluids, i.e., silicone compounds containing various combinations of elastomers in a variety of diluents. Examples of suitable cosmetic fluids are cyclopentasiloxane and amino propyldimethicone (Cosmetic fluid 1486-NH) (manufactured by Chemisil), cyclomethicone and dimethicone (Cosmetic Fluid 1684-DM) (manufactured by Chemisil), and a blend of low and high viscosity polydimethylsiloxane (e.g. Dow Corning 1413 Fluid™) (Dow Corning). Preferred is a blend of high viscosity polydimethylsiloxane in low viscosity polydimethylsiloxane (e.g. Dow Corning 1413 Fluid™) (Dow Corning).

In one embodiment the cosmetic composition is nonpigmented.

In one embodiment the cosmetic compositions contain one or more pigments, which are typically present in a different phase from the gel phase. The pigment used herein can be inorganic and/or organic. Cosmetic compositions according to the invention comprise greater than or equal to 0.1% pigments by weight of the cosmetic composition to provide a pigmenting effect. Typically, the pigments may be present from about 0.1% to 25%, preferably from about 0.5 to 15%, and most preferably from about 1 to 10% by weight The pigments are not fractal particles in accordance with the invention because they do not have the proper size domain, do not have the proper dimensionality, or are not charged particles. As used herein the term “pigments” includes lakes, and a single pigment or pigment combinations. Other colorants such as D&C dyes and self-tanning agents such as carbonyl derivatives or food colorants such as dihydroxyacetone (DHA) or erythrulose may be used. Pigments and colorants are used interchangeably herein.

Preferably, the pigments are selected from the group consisting of titanium oxides such as rutile titanium dioxide, anatase titanium dioxide, zinc oxide, zirconium oxide, iron oxides such as ferric oxide, ferrous oxide, yellow iron oxide, red iron oxide, bismuth oxy chlorides, black iron oxide, acylglutamate iron oxides, chromium oxide, chromium hydroxide, manganese violet, cerium oxide, ultramarine blue, carmine, and derivatives and mixtures thereof. More preferably, the pigment is titanium oxide, yellow iron oxide, red iron oxide, black iron oxide, and mixtures thereof. The pigments can be surface modified to render them either hydrophobic or hydrophilic to interact synergistically with the fractal particle network.

The cosmetic composition may also include opacifying agents (pearlescent agents) to add optical shimmer and luster or for tactile silkiness to the touch such as, but not limited to mica, sericite (a fine grained variety of muscovite). These agents may be present in amounts from about 0.1-10%, preferably about 0.5-5%.

The cosmetic composition may also include oil phase solvents useful as base fluids for spreading and lubrication properties or as a vehicle to provide a medium for one or more of the other constituents of the cosmetic composition. Solvents include water, organic fluids, especially alcohols and hydrocarbon fluids, silicone fluids, hydrophilic and hydrophobic polymers, and the like, and may be present in a concentration of about 0.5-90%, preferably about 5-50%, most preferably 10-35%. Preferred oil phase solvents are cyclomethicones such as cyclotetrasiloxane (e.g. Cyclo-2244 Cosmetic Grade Silicone (D4) (manufactured by Clearco), cyclopentasiloxane (e.g. Cyclo-2245 Cosmetic Grade Silicone (D5) (manufactured by Clearco), a cyclopentasiloxane/cyclohexasiloxane blend (D5/D6 Blend) Cyclo-2345 Cosmetic Grade Silicone (manufactured by Clearco), and a cyclomethicone/dimethiconol blend (D5/D4 Blend) Cyclo-1400 Cosmetic Grade Silicone (manufactured by Clearco). More preferred is D5.

Water typically is present in amounts ranging from about 10% to about 95% water by weight of the composition, preferably from about 40% to about 80%, and most preferably from about 40% to about 70%. Also suitable as aqueous phase solvents are low molecular weight alcohols having less than 8 carbons, for example ethanol, propanol, hexanol, and the like, and polyhydric alcohols, especially glycols. Suitable glycols are propylene glycol, pentylene glycol, hexylene glycol, and 1,2-octanediol. Suitable polyhydric alcohols include sorbitol and glycerin. These may be present in amounts of from about 1% to about 50%, preferably 5% to 35% by weight.

In another embodiment the compositions are substantially anhydrous suspensions of the macroscopic particles and the fractal particles in accordance with the present invention. Preferably, the fractal particles are fumed alumina, fumed silica, fumed titania, or combinations

Optionally, electrolytes such as sodium chloride may be added in amounts ranging from about 0-5%, preferably from about 0.5-2%.

The compositions of the invention further typically contain an amount of a pH adjusting agent to provide the desired pH of the composition, e.g., the pH at which the fractal particles will have the requisite opposite net surface charges. Suitable pH adjusting agents are organic and mineral acids as is well known in the cosmetic arts. Buffers to maintain the established pH may also be incorporated, for example sodium lactate.

It is further understood that the other cosmetic actives and adjuvants introduced into the composition must be of a kind and quantity that are not detrimental to the advantageous effect which is sought herein according to the invention.

The composition of the present invention improves the optical properties of films of cosmetic composition (as compared to those which merely reflect light producing a shiny appearance, those which merely cover the skin and impart a white cast to the skin, or those which either result in optical blurring or space filling, but not both). The resulting composition when applied to the skin, makes the skin appear more youthful, smoother and even in tone.

The physical arrangement of the gel structure, high particle loading and network formation, provides a smooth surface for topcoat (optical layer) applications of any foundation. The optical layer provides a unique “light releasing” effect from the skin when used as a primer for pigmented cosmetics. The optical layer mimics and enhances the skin's natural transparent qualities. When light penetrates the optical layer, diffuse reflection through the pigmented layer provides a “back lighting” effect, brightening foundations to give a more natural and youthful look

### Methods of Use

The methods of use for the cosmetic compositions disclosed and claimed herein concern the improvement in the aesthetic appearance of skin and include, but are not limited to: methods of blurring or masking one or more of wrinkles, fine lines, pores, skin imperfections, especially in the facial, neck or on or around the lip areas; methods to correct imperfections in skin such as blotches, freckles, redness, spider veins, and dark rings around the eyes; methods of enhancing or modifying skin color; and methods to improve finished makeup, and methods for application to the hair, eyelashes, and eyebrows.

The compositions of the present invention are suitable for use as a hair cosmetic, in particular as a mascara, in light of the unique rheological properties exhibited by the gels, as mentioned above. Thus, the compositions of the invention are free-flowing under shear, which allows them to be applied with a brush or suitable applicator. When the shear is removed the compositions return rapidly to the more viscous gel condition. Because the compositions are fractal, that is, they are porous, reticulated structures capable of maintaining geometric shape, they are able to coat hair and provide a volumizing benefit. Accordingly, they are ideal as mascaras, especially when formulated with a film former (as previously described), and as hair volumizers for treating thinning hair.

Examples of facial lines and skin imperfections which can be improved using the fractal gels of the present invention include, but are not limited to; frown lines that run between the eyebrows known as glabellar lines; perioral or smoker's lines which are vertical lines on the mouth; marionette lines at the corner of the mouth known as oral commissures; worry lines that run across the forehead; crow's feet at the corner of the eyes known as periorbital lines; deep smile lines that run from the side of the nose to corners of the mouth known as nasolabial furrows; cheek depressions; acne scars; some facial scars; wound or burn scars; keloids; to reduce dark rings around the eyes; to reduce the appearance of pores or blemishes, age spots, moles, birthmarks; to redefine the lip border; for artificial or self-tanning, and to reduce skin color unevenness or dullness.

In another embodiment the resulting gel can be employed as it is and can itself constitute a skin care or make-up composition for blurring wrinkles, fine lines, pores, and skin imperfections.

Therefore, the gels may comprise from about 1% to about 100% by weight, relative to the total weight of the composition.

Facial lines and wrinkles can be present anywhere on the face, and occur most frequently on the lips and in the eye area. However, it is understood by those skilled in the art that the composition can be applied to any part of the body where a blurring effect is desired such as to reduce wrinkles, fine lines, poses and skin imperfections. Non-limiting examples include to conceal imperfections in the skin, such as to mask the appearance of cellulite or vitiligo, to mask the appearance of spider vessels, moles, age spots, blemishes, scars, freckles, birth marks and varicose veins, to conceal damage incurred to the skin as a result of trauma such as cosmetic surgery, burns, stretching of skin, to conceal the appearance of villus hair on the skin; to provide UV protection to the skin.

The compositions herein can be used by topically applying to the areas of the skin an effective amount of the compositions. The effective amount can easily be determined by each user. As used herein the term “effective amount” refers to an amount sufficient to result in “optical blurring” of the appearance of the skin.

The composition can be applied for several days, weeks, months or years at any intervals. The compositions are generally applied by light massaging the composition onto the skin. However, the method of application may be any method known in the art and is thus not limited to the aforementioned.

The invention also relates to a method for therapeutic treatment of the skin. It is further understood that the gel of the present invention may be used together with therapeutic agents together with or adjunctive to pharmaceutical compositions including, but not limited to, anti-acne agents, sunscreens, self-tanning ingredients, anti-inflammatory agents, anti-bacterials, anti-fungals, anti-virals, anti-yeast agents, eye treatments, age spot treatments, analgesics, antidandruff and antiseborrhetic agents, hyperkeratolytics, antipsoriatic agents, skin lightening agents, depigmenting agents, wound healing agents, burn treatments, tanning agents, hair treatment agents, hair growth products, wart removers, antipuretics, and hormones.

### Preparation

Sol-gel chemistry techniques can be used to effect the formation of the gels of the present invention, as described in C. J. Brinker and W. Scherer, Acad. Press, Boston, 1990, which is incorporated by reference in its entirety herein.

The compositions useful for the methods of the present invention are generally prepared by conventional methods such as are known in the art of making topical cosmetic compositions. Such methods typically involve mixing of the ingredients in one or more steps to a relatively uniform state, with or without heating, cooling, application of vacuum, and the like.

Typically, the network of fractal particles is made by preparing a dispersion of each fractal particle in a suitable solvent (dispersant), adjusting the dispersion pH with a pH adjusting agent, if needed, e.g., when the fractal network is a fractal gel, and admixing the dispersions with shear to permit the formation of the fractal network. In some instances owing to the properties of the constituents it may be necessary to preheat the dispersant. Where fractal gels are being used to form the fractal network, the pH adjusting agent may also be provided into the admixed dispersions rather than into each dispersion individually. The macroparticles may then be incorporated into the dispersion, along with any actives and adjuvants that are desired. Certain of the adjuvants may require addition as premixes with a solvent, as generally known in the cosmetic art. The resulting gel can be employed as it is and can itself constitute a skin care or make-up composition for blurring wrinkles and skin imperfections.

Alternatively, the fractal gel may be incorporated into a multiphase cosmetic composition as previously mentioned. The other phase may be prepared in accordance with known methods, for example forming one or more premixes of the ingredients for combination with the gel. As previously mentioned the polymer in whole or in part may be incorporated into this other phase. Where premixes have been formed at elevated temperatures appropriate cooling of the composition to establish the emulsion will be necessary.

The following examples describe specific aspects of the invention to illustrate the invention and provide a description of the present methods for those skilled in the art. The Examples should not be construed as limiting the invention as the examples merely provide specific methodology useful in the understanding and practice of the invention and its various aspects.

### EXAMPLES

### General Example

The present invention is further illustrated by the following nonlimiting example.

TABLE 1
Illustrative Compositions

| Component | WEIGHT % |
|---|---|
| Polydimethylsiloxane and Cetearyl Dimethicone | 5-50 |
| Crosspolymer¹ | |
| Cellulose Beads (spherical) | 1-20 |
| Fumed Alumina (SpectrAL 51 from Cabot) | 0.1-10 |
| Amino Phenyltrimethicone | 0.1-10 |
| Ethylhexylmethoxy Cinnamate | 0.1-7.5 |
| Acrylates/Dimethicone copolymer/cyclomethicone | 0.1-10.0 |
| Decamethyl cyclopentasiloxane | 0.1-30 |
| Fragrance | 0-1 |
| Fumed Silica (Cabosil EH-5 from Cabot) | 0.1-10 |
| Preservative | 0.1-2 |
| Nylon Powder (spherical) | 0.1-10 |
| HDI/Trimethylol Hexyllactone Crosspolymer and Silica² | 0.1-10 |
| Polyethylene 1-20 um³ (spherical) | 0.1-10 |
| Boron Nitride (spherical) | 0.1-10 |
| Ethylhexyl Salicylate | 0.1-5 |
| POE (24M) Cholesterol Ether⁴ | 0.1-5 |
| Lauryl PEG-9 Polydimethylsiloxane Dimethicone⁵ | 0.1-5 |

¹Available from Dow Corning under the tradename DC 9041.
²Available from Kobo Products under the tradename BPD 800.
³Available from Presperse under the tradename Micropoly 220L.
⁴Available from Croda under the tradename Crodalan C24.
⁵Available from Shin Etsu under the tradename KF6038.

In the method of making of the preferred compositions of the present invention, the gel emulsifying agent, and sunscreens are premixed in a vessel. To a separate vessel, the solvent, film formers, wax, and preservative are added and heated to 180 to 190 degrees F. with mixing. Once the temperature is constant and the materials are well mixed, the fumed alumina and silica are added. Mixing continues until all of the fumed material is evenly dispersed. The premixed gel phase is then added to the solvent/film former/wax mixture. Mixing continues for 10 to 60 minutes, as the batch cools the remaining powdered components are added. Fragrance is added when the temperature is below 120° F.

When wax is used as a structurant in the composition the processing temperature is maintained above the solidification point of the wax and a hot fill is used.

TABLE 2
Illustrative Composition Containing Wax

| Component | WEIGHT % |
|---|---|
| Polydimethylsiloxane and Cetearyl Dimethicone | 5-50 |
| Crosspolymer¹ | |
| Cellulose Beads (spherical) | 1-20 |
| Fumed Alumina (SpectrAL 51 from Cabot) | 0.1-10 |
| Ethylhexylmethoxy Cinnamate | 0.1-7.5 |
| Acrylates/Dimethicone copolymer/cyclomethicone | 0.1-10.0 |
| Decamethyl cyclopentasiloxane | 0.1-30 |
| Fragrance | 0-1 |
| Fumed Silica (Cabosil EH-5 from Cabot) | 0.1-10 |
| Preservative | 0.1-2 |
| Nylon Powder (spherical) | 0.1-10 |
| HDI/Trimethylol Hexyllactone Crosspolymer and Silica² | 0.1-10 |
| Polyethylene 1-20 um³ (spherical) | 0.1-10 |
| Boron Nitride (spherical) | 0.1-10 |
| Ethylhexyl Salicylate | 0.1-5 |
| C30-45 Alkyl Methicone/C30-45 Olefin | 0.1-5 |
| Lauryl PEG-9 Polydimethylsiloxane Dimethicone⁵ | 0.1-5 |

¹Available from Dow Corning under the tradename DC 9041.
²Available from Kobo Products under the tradename BPD 800.
³Available from Presperse under the tradename Micropoly 220L.
⁵Available from Shin Etsu under the tradename KF6038.

Skin care compositions of the present invention are found to reduce the visibility of wrinkles to a greater extent than skin care products not containing the gels of the present invention.

### Working Examples

### Without Pigment

Examples I through VI are illustrative of the present invention in which Examples I-III are aqueous emulsions and Examples IV-VI are anhydrous compositions, wherein the fractal particles 3, 4 and 5 are dispersed in D5.

TABLE 3
Colorless Primer Compositions

| | | I | II | III | IV | V | VI |
|---|---|---|---|---|---|---|---|
| 1 | Dow Corning 1413 Fluid | 10 | 10 | 10 | 15 | 15 | 15 |
| 2 | Velvesil 125 | 30 | 30 | 30 | 35 | 30 | 30 |
| 3 | Fumed Silica | 0 | 0 | 0 | 7 | 10 | 7 |
| 4 | Fumed TiO2 | 0 | 0 | 0 | 0 | 5 | 8 |
| 5 | Fumed Alumina | 0 | 0 | 0 | 10 | 5 | 5 |
| 6 | Nylon | 0 | 0 | 0 | 3 | 5 | 5 |
| 7 | Dow Corning 5225 C | 0 | 0 | 9 | 0 | 0 | 0 |
| 8 | D5 | 15 | 15 | 9 | 30 | 30 | 30 |
| 9 | RM 2-2051 | 4 | 4 | 0 | 0 | 0 | 0 |
| | (Dow Corning) | | | | | | |
| 10 | Water | 11 | 11 | 11 | 0 | 0 | 0 |
| 11 | 30% Silica/Alumina (1:1) | 0 | 30 | 15 | 0 | 0 | 0 |
| | dispersion in Water | | | | | | |
| 12 | 30% Silica/Alumina (2:1) | 30 | 0 | 15 | 0 | 0 | 0 |
| | dispersion in Water | | | | | | |
| 13 | NaCl | 0 | 0 | 1 | 0 | 0 | 0 |
| | Total | 100 | 100 | 100 | 100 | 100 | 100 |

1 Dimethicone (3300 cs.) sold by Dow Corning Corp. as DC 1413 fluid
2 Macroscopic dimethicone/vinyl dimethicone crosspolymer available from Momentive Performance Materials, Inc.
3 Cabosil EH-5 available from Cabot Company
4 Aeroxide P25 from Degussa
5 SpectrAl 51 from Cabot
6 Orgasol 2002 B Natural Extra COS from Lipa Chemical
7 Cyclopentasiloxane and PEG/PPG-18/18 Dimethicone sold by Dow Corning Corp.
8 Cyclomethicone available from Dow Corning under the tradename DC 245 Fluid
9 Thickening agent containing sodium polyacrylate, dimethicone, cyclopentasiloxane, trideceth-6 and PEG/PPG-18/18 dimethicone sold by Dow Corning Corp.
11 and 12 are fractal particle gel dispersions containing fumed silica (Cabosil EH-5) and fumed alumina (SpectrAl 51) both sold by Cabot Corporation in the proportions indicated

The compositions I through III were made as follows:

Phase A—Silicone Phase: Dow Corning 1413 Fluid, Velvesil 125, DC 5225 C were mixed until homogeneous followed by addition of D5 and Nylon using a 3 blade propeller mixer. RM 2051 was then added to the silicone phase and mixed for an additional 10 minutes.

Phase B—Aqueous phase: A 30 wt % dispersion of fumed silica/alumina was added to water and NaCl until homogenous for about 20 minutes at room temperature. Phase B was then slowly added to phase A with continuous stirring for 10 minutes. The composition was then mixed further for an additional 20 minutes.

The compositions IV through VI were made as follows:

Velvesil 125, D5, and (1413 Fluid are added and heated to 180 to 190° F. with mixing. Once the temperature is constant and the materials are well mixed, the fumed alumina and silica are added. Mixing continues until all of the fumed material is evenly dispersed. Mixing continues for 10 to 60 minutes, as the batch cools the remaining powdered components (nylon) are added.

### Working Examples with Pigment

The following examples are illustrative of a foundation composition containing pigments.

TABLE 4
Foundation Compositions

| | | I | II | III | IV | V |
|---|---|---|---|---|---|---|
| 1 | TiO2 | 5 | 4 | 3 | 5 | 5 |
| 2 | Red Iron Oxide | 2 | 3 | 2 | 2 | 3 |
| 3 | Yellow Iron Oxide | 3 | 2 | 2 | 3 | 2 |
| 4 | Black Iron Oxide | 1 | 1 | 1 | 1 | 1 |
| 5 | Sericite | 5 | 4 | 5 | 3 | 3 |
| 6 | Dow Corning 1413 Fluid | 10 | 10 | 10 | 10 | 12 |
| 7 | Velvesil 125 | 25 | 25 | 25 | 30 | 27 |
| 8 | Fumed Silica | 0 | 0 | 0 | 8 | 7 |
| 9 | Fumed TiO2 | 0 | 0 | 0 | 5 | 8 |
| 10 | Fumed Alumina | 0 | 0 | 0 | 5 | 2 |
| 11 | Nylon | 0 | 0 | 0 | 5 | 5 |
| 12 | Dow Corning 5225C | 0 | 0 | 10 | 0 | 0 |
| 13 | D5 | 10 | 10 | 9 | 23 | 25 |
| 14 | Rm 2-2051 (Dow Corning) | 4 | 4 | 0 | 0 | 0 |
| 15 | Water | 5 | 7 | 8 | 0 | 0 |
| 16 | 30% Silica/Alumina | 0 | 30 | 12 | 0 | 0 |
| | (1:1) dispersion in | | | | | |
| | Water | | | | | |
| 17 | 30% Silica/Alumina | 30 | 0 | 12 | 0 | 0 |
| | (2:1) dispersion in | | | | | |
| | Water | | | | | |
| 18 | NaCl | 0 | 0 | 1 | 0 | 0 |
| | Total | 100 | 100 | 100 | 100 | 100 |

6 Dimethicone (3300 cs.) sold by Dow Corning Corp. 1431 Fluid is the trade name
7 Macroscopic dimethicone/vinyl dimethicone crosspolymer available from Momentive Performance Materials, Inc.
8 Cabosil EH-5 available from Cabot Company
9 Aeroxide P25 from Degussa
10 SpectrAL 51 from Cabot
11 Orgasol 2002 B Natural Extra COS from Lipa Chemical
12 Cyclopentasiloxane and PEG/PPG-18/18 Dimethicone sold by Dow Corning Corp.
13 Cyclomethicone available from Dow Corning under the tradename DC 245 Fluid
14 Thickening agent containing sodium polyacrylate, Dimethicone, Cyclopentasiloxane, Trideceth-6 and PEG/PPG-18/18 Dimethicone sold by Dow Corning Corp.
16 and 17 are fractal particle gel dispersions containing fumed silica (Cabosil EH-5) and fumed alumina (SpectrAl 51) both sold by Cabot Corporation in the proportions indicated

Examples I-III are aqueous emulsions; Examples IV-V are anhydrous compositions in which the fractal particles 8, 9 and 10 are dispersed in D5.

The present invention provides a variety of compositions useful in solid and/or semi-solid forms (including creams, gels and viscous liquids). Such compositions are preferably foundations, but also include face sticks, pancakes, and other facial cosmetic products.

Although the present invention describes in detail certain embodiments, it is understood that variations and modifications may exist that are known to those skilled in the art but, nonetheless, fall within the scope of the present invention. Accordingly, the present invention is intended to encompass all such alternatives, modification and variations that are within the scope of the invention as set forth in the following claims.

The contents of all patents, patent applications, published PCT applications and articles, books, references, reference manuals and abstracts cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

1. A cosmetic composition comprising a gel system comprising (a) a fractal network of nanoparticles; and (b) translucent macroscopic particles.

2. The cosmetic composition of claim 1 wherein the nanoparticles are inorganic nanoparticles having a particle size of between about 50 to 900 nm and a refractive index of from about 1.38 to about 2.

3. The cosmetic composition of claim 2 wherein the inorganic nanoparticles are selected from the group consisting of silica, alumina, titania, zirconia, zinc oxide, indium tin oxide, ceria, and mixtures thereof, and wherein the macroscopic particles are selected from the group consisting of silicone elastomers; silicone crosspolymers; hydrocarbon elastomers; natural and synthetic rubbers; polymeric spheres, and compatible combinations thereof.

4. The composition of claim 3 wherein the polymeric spheres are selected from the group consisting of fluoropolymers, polyacrylates, nylon, polyesters, cellulose beads, polyurethanes, polyacrylic esters, polyethers, polyamides, polyesteramides, polyurethanes, and mixtures thereof.

5. The cosmetic composition of claim 3 wherein the gel is present in an amount of from about 1 to 100% by weight of the composition.

6. The cosmetic composition of claim 4 wherein the gel contains at least about 2% and less than 60% nanoparticles by weight of the gel.

7. The cosmetic composition of claim 4 wherein the macroparticles are selected from the group consisting of silicone elastomers; silicone crosspolymers; nylon; polyurethane; cellulose beads, and mixtures thereof.

8. The cosmetic composition of claim 7 wherein the macroscopic particles are selected from the group consisting of cross-linked silicone elastomers derived from vulcanizable silicone sealant chemistry, addition-polymerized silicone elastomers prepared by the hydrosilylation of olefins or olefinic silicones with silyl hydrides, and silicone crosspolymers obtained by self-polymerization of bifunctional precursor molecules containing epoxy-silicone and silyl hydride functionalities in the absence of crosslinker molecules.

9. The cosmetic composition of claim 8 wherein said silicone crosspolymers are selected from the group consisting of dimethicone/vinyl dimethicone crosspolymers, vinyl dimethicone/lauryl dimethicone crosspolymers, alkyl ceteayl dimethicone/polycyclohexane oxide crosspolymers, and mixtures thereof.

10. The cosmetic composition of claim 7 wherein the macroscopic particles have a refractive index range of from about 1.38 to about 1.6.

11. The cosmetic composition of claim 10 wherein the gel is present in an amount of from about 5 to about 99% and wherein the cosmetic composition further comprises an active agent.

12. The cosmetic composition of claim 11 wherein the active agent is selected from the group consisting of pigments, light diffusers, sunscreens, uv light absorbers, and compatible combinations thereof.

13. The cosmetic composition of claim 1 wherein different nanoparticles form different fractal networks.

14. The cosmetic composition of claim 3 wherein the gel is an aqueous dispersion of first nanoparticles and second nanoparticles having a predetermined pH, the first and second nanoparticles having oppositely charged zeta potentials at said pH.

15. The cosmetic composition of claim 14 wherein the first nanoparticle is alumina and the second nanoparticle is silica.

16. The cosmetic composition of claim 3 wherein the cosmetic composition is substantially anhydrous and the gel further comprises a nonaqueous solvent.

17. The cosmetic composition of claim 3 further comprising a solvent in which the macroscopic particle is dispersed, and wherein the refractive index of the fractal particle does not match the refractive index of the macroscopic particles.

18. A cosmetic composition comprising:
a) an aqueous gel fractal network having a predetermined pH present in an amount of from about 3 to about 90% by weight of the cosmetic composition comprising a first nanoparticle and a second nanoparticle, the first and second nanoparticles having oppositely charged zeta potentials at said pH, said first and second nanoparticles being selected from the group consisting of alkyl substituted fumed silica, fumed silica, colloidal silica, fumed alumina, fumed titania, and mixtures thereof;
b) translucent macroscopic particles having a particle size of from about 1 to about 200 microns and a refractive index of from about 1.38 to about 1.6, the refractive index of the nanoparticles not matching the refractive index of the macroscopic particles;
c) at least one cosmetic active ingredient.

19. The cosmetic composition of claim 18 wherein said nanoparticles have a particle size of about 50 to about 200 nm and wherein the macroscopic particles have a particle size of between about 2 to about 50 microns.

20. The cosmetic composition of claim 19 wherein the macroscopic particles are selected from the group consisting of silicone elastomers, hydrocarbon elastomers, silicone crosspolymers, nylon, polyurethane, cellulose beads, and combinations thereof.

21. A substantially anhydrous cosmetic composition comprising a gel comprising (a) a fractal network of nanoparticles; (b) translucent macroscopic, and (c) a nonaqueous solvent, the refractive index of the nanoparticles not matching the refractive index of the macroscopic particles.

22. The cosmetic composition of claim 21 wherein the nanoparticles are selected from the group consisting of alkyl substituted fumed silica, fumed silica, colloidal silica, fumed alumina, fumed titania, and mixtures thereof.

23. The cosmetic composition of claim 22 wherein the macroparticles are selected from the group consisting of silicone elastomers, hydrocarbon elastomers, silicone crosspolymers, polymeric spheres, and combinations thereof.

24. The cosmetic composition of claim 21 wherein the macroparticles are selected from the group consisting of silicone elastomers, hydrocarbon elastomers, silicone crosspolymers, polymeric spheres, and combinations thereof.

25. The cosmetic composition of claim 24 wherein the polymeric spheres are selected from the group consisting of fluoropolymers, polyacrylates, nylon, polyesters, cellulose beads, polyurethanes, polyacrylic esters, polyethers, polyamides, polyesteramides, polyurethanes, and mixtures thereof.

26. The cosmetic composition of claim 21 wherein said nanoparticles have a particle size of about 50 to about 200 nm, and wherein the macroscopic particles have a particle size of between about 2 to about 50 microns and a refractive index of from about 1.38 to about 1.6.

27. The cosmetic composition of claim 21 wherein the solvent is selected from the group consisting of hydrocarbon fluids and silicone fluids.

28. A gel system comprising translucent macroparticles within a fractal network of nanoparticles.

29. The gel system of claim 28 wherein the refractive index of the nanoparticles do not match the refractive index of the macroscopic particles.

30. The gel system of claim 29 wherein the fractal network is an aqueous dispersion of first nanoparticles and second nanoparticles having a predetermined pH, the first and second nanoparticles having oppositely charged zeta potentials at said pH and wherein the macroscopic particles are at least translucent.

31. The gel system of claim 29 wherein the fractal network is a substantially anhydrous dispersion of nanoparticles in a nonaqueous solvent.

32. A method for optically blurring the appearance of skin imperfections selected from the group consisting of wrinkles, fine lines, and pores comprising the step of applying to the skin an amount of a skin care or make-up composition effective to optically blur the appearance of said skin imperfection, the composition comprising a gel system comprising (a) a fractal network of nanoparticles; and (b) translucent macroscopic particles.
